(19) United States
(12) Patent Application Publication
(10) Pub. No.: US 2022/0177912 A1
(43) Pub. Date: Jun. 9, 2022

(54) THERAPEUTIC PARTICLES THAT ENABLE ANTIGEN PRESENTING CELLS TO ATTACK CANCER CELLS

(71) Applicant: The Methodist Hospital, Houston, TX (US)

(72) Inventors: Haifa Shen, Houston, TX (US); Gang Li, Houston, TX (US)

(73) Assignee: The Methodist Hospital, Houston, TX (US)

(21) Appl. No.: 17/601,812

(22) PCT Filed: Apr. 13, 2020
(86) PCT No.: PCT/US20/27968

§ 371 Date: Oct. 6, 2021

Related U.S. Application Data
(60) Provisional application No. 62/833,110, filed on Apr. 12, 2019.

(51) Int. Cl. C12N 15/85; A61K 35/15; C07K 16/30; C07K 14/705; A61P 35/00

(52) U.S. Cl. CPC C12N 15/85; A61K 35/15; C07K 16/30; C07K 14/70521; C07K 2319/03; C07K 16/3015; A61P 35/00; C07K 2317/622; C07K 14/70578

(57) ABSTRACT

Embodiments of the present disclosure pertain to modified antigen presenting cells that include a recombinant protein appended onto a surface of the antigen presenting cells. The recombinant protein can include: an ectodomain positioned on the surface; a transmembrane domain with a region embedded in the cell membrane; an antigen presenting cell recruiting domain that directs the cells towards the cancer cells; and an antigen presenting cell activator that activates or licenses the antigen presenting cells. Additional embodiments of the present disclosure pertain to methods of expressing the recombinant proteins on antigen presenting cells and utilizing the modified antigen presenting cells for treating various cancers in various subjects. Further embodiments of the present disclosure pertain to nucleotide-containing carriers for expressing the recombinant proteins of the present disclosure in antigen presenting cells.

### CROSS-REFERENCE TO RELATED APPLICATIONS

This application claims priority to U.S. Provisional Patent Application No. 62/833,110, filed on Apr. 12, 2019. The entirety of the aforementioned application is incorporated herein by reference.

### BACKGROUND

Current methods of utilizing chimeric antigen receptor cells to treat cancer have numerous limitations, including multiple and prolonged steps, a need for the identification of highly specific antigens, undesired side effects (e.g., side effects associated with off-target effects), and the need for the utilization of adjuvants. Numerous embodiments of the present disclosure address the aforementioned limitations.

### SUMMARY

In some embodiments, the present disclosure pertains to modified antigen presenting cells that include a recombinant protein appended onto a surface of the antigen presenting cells. In some embodiments, the recombinant protein includes an ectodomain that is positioned on the surface of the antigen presenting cells and capable of preferentially binding to an antigen of cancer cells. The recombinant protein also includes a transmembrane domain that includes at least one region embedded in the antigen presenting cell membrane that serves as an anchor for maintaining the ectodomain of the recombinant protein on the surface of the antigen presenting cells.

In additional embodiments, the recombinant protein also includes an antigen presenting cell-recruiting domain that directs the antigen presenting cells towards the cancer cells. In further embodiments, the recombinant protein also includes an antigen presenting cell activator that activates or licenses the antigen presenting cells.

In additional embodiments, the present disclosure pertains to methods of expressing the recombinant proteins of the present disclosure on antigen presenting cells by introducing a carrier into the antigen presenting cells. The carrier includes a nucleotide sequence. The recombinant protein is expressed by the antigen presenting cells from the nucleotide sequence and appended onto a surface of the antigen presenting cells.

The methods of the present disclosure can have numerous in vitro and in vivo embodiments for various purposes. For instance, in some embodiments, the methods of the present disclosure can include administering the carriers of the present disclosure into a subject to result in the preferential uptake of the carriers by antigen presenting cells and the expression of the recombinant proteins of the present disclosure on the surfaces of the antigen presenting cells. Thereafter, the modified antigen presenting cells can bind to cancer cells through an interaction between the recombinant proteins and cancer cells, thereby activating an anti-cancer immune response against the cancer cells.

In alternative embodiments, the methods of the present disclosure can include a step of introducing the carriers of the present disclosure into antigen presenting cells to result in the expression of the recombinant proteins of the present disclosure on the surfaces of the antigen presenting cells. Thereafter, the modified antigen presenting cells are introduced to a subject in order to activate an anti-cancer immune response against the cancer cells in the subject.

The methods of the present disclosure can have numerous applications. For instance, in some embodiments, the methods of the present disclosure can be utilized to treat a cancer in a subject by introducing the carriers or modified antigen presenting cells of the present disclosure to the subject. The methods of the present disclosure can be utilized to treat numerous types of cancers in subjects. For instance, in some embodiments, the cancer includes cancers associated with epithelial derived solid tumors, bone marrow-derived tumors, and combinations thereof.

Additional embodiments of the present disclosure pertain to the carriers of the present disclosure. In some embodiments, the carriers of the present disclosure are in the form of particles that encapsulate nucleotides that express the recombinant proteins of the present disclosure.

### DESCRIPTION OF THE DRAWINGS

FIG. 1A illustrates an antigen presenting cell in accordance with an embodiment of the present disclosure.

FIG. 1B illustrates an in vivo method of modifying antigen presenting cells and utilizing the modified antigen present cells to treat a cancer in a subject.

FIG. 1C illustrates an in vitro method of modifying antigen presenting cells and introducing the modified antigen present cells to a subject for the treatment of cancer.

FIG. 1D illustrates a method of treating cancer by introducing the modified antigen presenting cells of the present disclosure to a subject.

FIG. 1E illustrates a carrier for introducing nucleotides that express the recombinant proteins of the present disclosure to antigen presenting cells.

FIG. 2 provides an illustration of a glycosylphosphatidylinositol (GPI)-anchored single chain variable fragment (scFv) modified dendritic cell (DC) (GPI anchored scFv modified DC).

FIG. 3 provides an illustration of a chimeric antigen receptor dendritic cell (CAR-DC) (FIG. 3A) and the nucleotide sequence that expresses the chimeric antigen receptor (FIG. 3B).

FIG. 4 provides cellular images indicating that GPI anchors in GPI anchored scFv modified DCs translocate recombinant proteins to cell membranes.

FIG. 5 provides cellular images indicating that CAR-DC recombinant proteins are located on cell membranes.

FIG. 6 provides data indicating that anti-EPCAM scFv in GPI anchored scFv modified DCs is human specific.

FIG. 7 provides additional data indicating that anti-EPCAM scFv in GPI anchored scFv modified DCs is human specific.

FIG. 8 provides additional data indicating that anti-EPCAM scFv in GPI anchored scFv modified DCs is human specific.

FIG. 9 provides data regarding the binding ability of CMV driven anti-EPCAM GPI expressing DC2.4 cells.

FIG. 10 provides additional data and images regarding the specificity of anti-EPCAM scFv in GPI anchored scFv modified DCs for human cells.

FIG. 11 provides additional data regarding the specificity of anti-EPCAM scFv in GPI anchored scFv modified DCs for human cells.

FIG. 12 provides additional data regarding the specificity of anti-EPCAM scFv in GPI anchored scFv modified DCs for human cells.

FIG. 13 provides additional data regarding the specificity of anti-EPCAM scFv in CAR-DCs for human cells.

FIG. 14 provides data illustrating the synergistic effects of TLR4 and CD40 in activating dendritic cells.

FIG. 15 provides additional data illustrating the synergistic effects of TLR4 and CD40 in activating dendritic cells.

FIG. 16 provides additional data illustrating the synergistic effects of TLR4 and CD40 in activating dendritic cells.

FIG. 17 provides data illustrating the anti-tumor potentials of CAR-DC and GPI anchored scFv modified DCs.

FIG. 18 provides additional data illustrating the anti-tumor potentials of CAR-DC and GPI anchored scFv modified DCs.

FIG. 19 provides additional data illustrating the anti-tumor potentials of CAR-DC and GPI anchored scFv modified DCs.

FIG. 20 provides additional data illustrating the anti-tumor potentials of CAR-DC and GPI anchored scFv modified DCs.

FIG. 21 provides additional data illustrating the anti-tumor potentials of CAR-DC and GPI anchored scFv modified DCs.

FIG. 22 provides additional data illustrating the anti-tumor potentials of CAR-DC and GPI anchored scFv modified DCs.

FIG. 23 provides additional data illustrating the anti-tumor potentials of CAR-DC and GPI anchored scFv modified DCs.

FIG. 24 provides images that illustrate the immunofluorescence of anti-Her2 GPI expressing DC 2.4 cells.

FIG. 25 provides data regarding the interaction of anti-Her2 GPI expressing DC 2.4 cells with cancer cells.

FIG. 26 provides an illustration of pDNA nanoparticles.

FIG. 27 provides data indicating that complexes (identified as PBAE-MTAS-NLS) from nanoparticles are able to trap pDNA.

FIG. 28 provides data indicating that both the core and shell components of pDNA nanoparticles are necessary for recombinant protein gene expression.

FIG. 29 provides additional data indicating that both the core and shell components of pDNA nanoparticles are necessary for recombinant protein gene expression.

FIG. 30 provides additional data indicating that both the core and shell components of pDNA nanoparticles are necessary for recombinant protein gene expression.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Chimeric antigen receptor (CAR) T cell therapy is an effective immunotherapy for some patients with certain kinds of cancers. By expressing an engineered receptor, T-cells could recognize cancer cells (through particular antigens on their surfaces), activate themselves (in a major histocompatibility complex (MHC) independent manner), proliferate, and motivate other components in the immune system (through the secretion of cytokines) to fine-tune the immune response against cancer cells.

The aforementioned effects are generally achieved through different domains of engineered receptors. For instance, the ectodomain of CAR is usually a single-chain variable fragment (scFv) that binds to a particular antigen, such as CD19 on B cell malignancies, which define the specificity and safety of CAR-T cell therapy.

CD3 zeta chain, the signal-transduction component of the T cell receptor (TCR)-CD3 complex, is the most important endodomain as it transmits an activation signal to the T cell after antigen-scFv binding. Other co-stimulatory domains, such as CD28 and 4-1BB, are effective in transmitting proliferative or survival signals to fully activate T-cells.

However, there are still many challenges to overcome for the applications of CAR-T in a broad range of cancer types, such as solid tumors. For instance, the heterogeneity of cancer cells makes it difficult to identify suitable antigens. Moreover, the non-specific antigen may compromise the safety and efficiency of the treatment. Furthermore, the efficacy of CAR-T is limited by the immunosuppressive cancer microenvironment, which is comprised of multiple immunosuppressive cell types, cytokines and suboptimal conditions such as hypoxia and acidification.

Many efforts have been made to reinforce the functions of CAR-T in the cancer microenvironments. Such efforts have included the design of more efficient CAR-T and their therapeutic combination with small molecules. However, only limited effectiveness has been observed.

Additionally, CAR-T treatment is time and effort consuming, making it too expensive for the majority of patients. Generally, T cells must first be collected from patients, genetically modified to express CAR, expanded for several weeks to obtain enough functional CAR-T cells, and then infused back to patients under lympho-depleting chemotherapy. These complicated procedures call for dedicated equipment and expertise, thereby making them difficult to apply on a large scale.

Additional limitations exist for the use of CAR-T. For instance, to obtain better therapeutic effects, many CAR-T therapies require the need for the utilization of adjuvants to activate the antigen presenting cells. Additionally, numerous side effects are associated with the administration of the chimeric antigen receptor cells and adjuvants to subjects.

As such, a need exists for modified antigen presenting cells that can be used to treat numerous types of cancers in various subjects in a cost effective and efficient manner. Numerous embodiments of the present disclosure address the aforementioned need.

In some embodiments, the present disclosure pertains to modified antigen presenting cells that include a recombinant protein appended onto a surface of the antigen presenting cells. In some embodiments, the recombinant protein includes an ectodomain that is positioned on the surface of the antigen presenting cells and capable of preferentially binding to an antigen of cancer cells. The recombinant protein also includes a transmembrane domain that includes at least one region embedded in the antigen presenting cell membrane that serves as an anchor for maintaining the ectodomain of the recombinant protein on the surface of the antigen presenting cells.

In additional embodiments, the recombinant protein also includes an antigen presenting cell recruiting domain that directs the antigen presenting cells towards the cancer cells. In further embodiments, the recombinant protein also includes an antigen presenting cell activator that activates or licenses the antigen presenting cells.

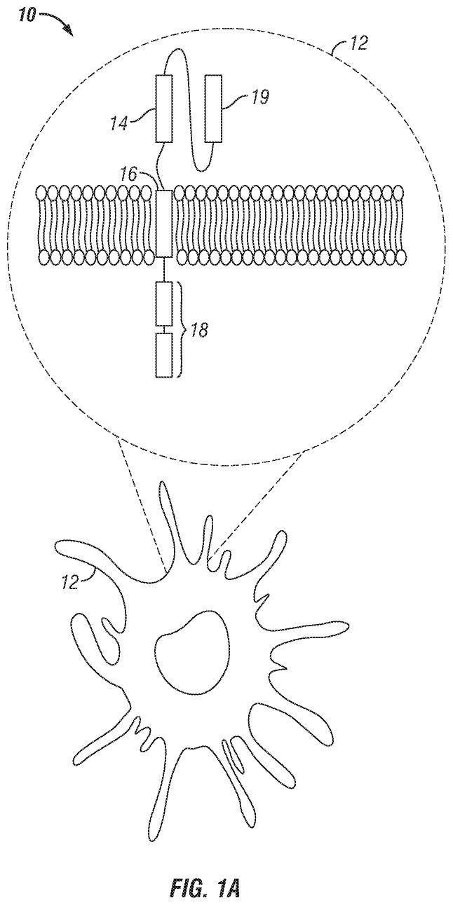

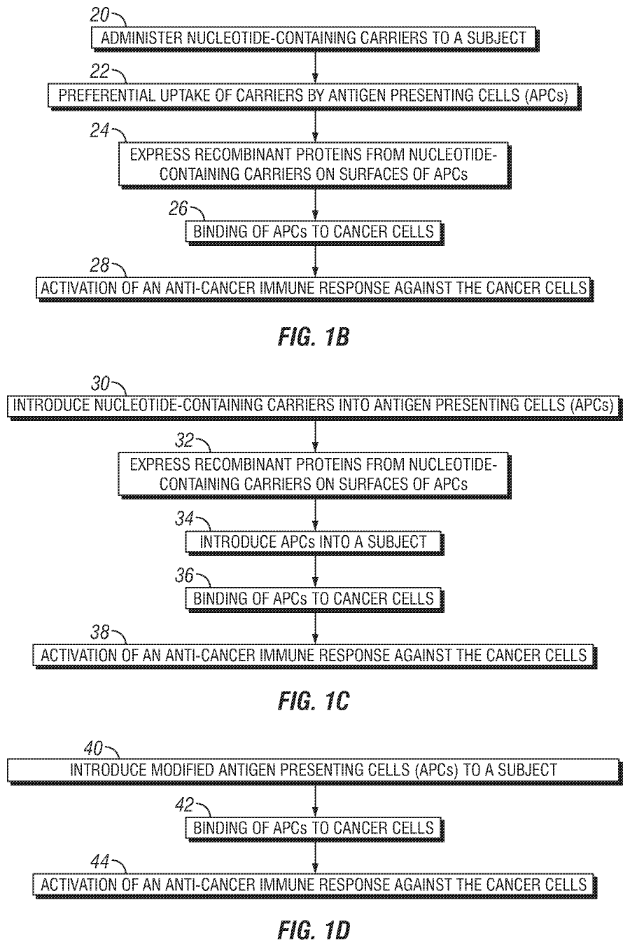

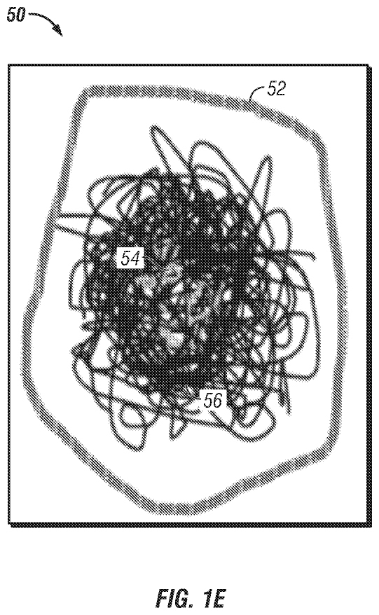

In some embodiments illustrated in FIG. 1A, the modified antigen presenting cells of the present disclosure are exemplified as an antigen presenting cell 10 with a recombinant protein appended onto its surface 12. The recombinant protein includes: (1) an ectodomain 14 that is positioned on surface 12 of antigen presenting cell 10 and capable of preferentially binding to an antigen of cancer cells; (2) a transmembrane domain 16 with at least one region embedded in the antigen presenting cell membrane that serves as an anchor for maintaining ectodomain 14 on surface 12; (3) an antigen presenting cell recruiting domain 19 that directs antigen presenting cell 10 towards cancer cells; and (4) antigen presenting cell activator 18 that activates or licenses antigen presenting cell 10.

In additional embodiments, the present disclosure pertains to methods of expressing the recombinant proteins of the present disclosure on antigen presenting cells by introducing a carrier into the antigen presenting cells. The carrier includes a nucleotide sequence. The recombinant protein is expressed by the antigen presenting cells from the nucleotide sequence and appended onto a surface of the antigen presenting cells.

The methods of the present disclosure can have numerous in vitro and in vivo embodiments for various purposes. For instance, as illustrated in FIG. 1B, the methods of the present disclosure can include administering the carriers of the present disclosure into a subject (step 20) to result in the preferential uptake of the carriers by antigen presenting cells (step 22) and the expression of the recombinant proteins of the present disclosure on the surfaces of the antigen presenting cells (step 24). Thereafter, the modified antigen presenting cells can bind to cancer cells through an interaction between the recombinant proteins and cancer cells (step 26), thereby activating an anti-cancer immune response against the cancer cells (step 28).

Likewise, as illustrated in FIG. 1C, the methods of the present disclosure can include a step of introducing the carriers of the present disclosure into antigen presenting cells (step 30) to result in the expression of the recombinant proteins of the present disclosure on the surfaces of the antigen presenting cells (step 32). Thereafter, the modified antigen presenting cells are introduced to a subject (step 34). The introduced modified antigen presenting cells can then bind to cancer cells in the subject through an interaction between the recombinant proteins and cancer cells (step 36), thereby activating an anti-cancer immune response against the cancer cells in the subject (step 38).

As further illustrated in FIG. 1D, the methods of the present disclosure also pertain to methods of treating a cancer in a subject by introducing the modified antigen presenting cells of the present disclosure to the subject (step 40) to result in the binding of the modified antigen presenting cells to cancer cells in the subject through an interaction between the recombinant proteins and cancer cells (step 42) and activation of an anti-cancer immune response against the cancer cells in the subject (step 44).

Additional embodiments of the present disclosure pertain to the carriers of the present disclosure. In some embodiments, the carriers of the present disclosure are in the form of particles that encapsulate nucleotides that express the recombinant proteins of the present disclosure. In some embodiments illustrated in FIG. 1E, the carriers of the present disclosure are in the form of particle 50. Particle 50 includes shell 52 that encapsulates nucleotides 54, which are embedded in polymers 56.

As set forth in more detail herein, the carriers, modified antigen presenting cells and methods of the present disclosure can have numerous embodiments. In particular, numerous types of recombinant proteins may be appended onto surfaces of numerous antigen presenting cells for interaction with various types of cancers in various subjects through various mechanisms.

Antigen Presenting Cells

The recombinant proteins of the present disclosure may be appended to numerous antigen presenting cells. For instance, in some embodiments, the antigen presenting cells include, without limitation, macrophages, B-cells, dendritic cells, and combinations thereof.

In some embodiments, the antigen present cells include dendritic cells. In some embodiments, the use of dendritic cells (DCs) as modified antigen presenting cells are advantageous because they link innate to adaptive immunity, thereby enabling the generation of anti-tumor immune responses against a variety of cancer cell antigens, despite of their heterogeneity.

For instance, in some embodiments, immature dendritic cells have a strong ability to uptake and present antigens, such as debris from cancer cells, and become mature under adverse signals in a tumor microenvironment. These mature dendritic cells can then home to lymph nodes and become licensed by antigen specific CD4⁺ T cells through CD40-CD40L interactions. Thereafter, licensed dendritic cells can activate cognate CD8⁺ T cells and trigger tumor regression or eradication.

Recombinant Proteins

In general, the recombinant proteins of the present disclosure include an ectodomain and a transmembrane domain. In additional embodiments, the recombinant proteins of the present disclosure also include an antigen presenting cell recruiting domain. In further embodiments, the recombinant proteins of the present disclosure also include an antigen presenting cell activator. As set forth in more detail herein, the recombinant proteins of the present disclosure include various ectodomains, transmembrane domains, recruiting domains, and antigen presenting cell activators.

Ectodomains

An ectodomain generally refers to a region of a recombinant protein that is positioned on the surface of the antigen presenting cells of the present disclosure. In addition, the ectodomain is capable of preferentially binding to an antigen of cancer cells.

The recombinant proteins of the present disclosure can include numerous ectodomains. For instance, in some embodiments, the ectodomains of the present disclosure include, without limitation, antibodies, single chain antibodies, nanobodies, aptamers, antibody fragments, portions of antibodies, scFV portions of antibodies, peptides, and combinations thereof. In some embodiments, the ectodomains of the present disclosure include an antibody fragment.

The ectodomains of the present disclosure can preferentially bind to numerous antigens of cancer cells. For instance, in some embodiments, the cancer cell antigen includes, without limitation, moieties, proteins, glycoproteins, EGF receptors, MUC-1, lipids, EPCAM, HER-2 receptors, and combinations thereof.

In some embodiments, the cancer cell antigens include an EPCAM protein, and the ectodomain is an anti-EPCAM antibody. In some embodiments, the cancer cell antigen is a HER-2 receptor, and the ectodomain is an anti-HER-2 antibody.

Transmembrane Domains

A transmembrane domain generally refers to a recombinant protein site that includes one region that is embedded in the antigen presenting cell membrane. The transmembrane domain also serves as an anchor for maintaining the ectodomain on the surface of the antigen presenting cells.

The recombinant proteins of the present disclosure can include numerous transmembrane domains. For instance, in some embodiments, the transmembrane domain includes, without limitation, glycosylphosphatidylinositol (GPI)-anchored domains, glycolipid-linked domains, CD8-based transmembrane domains, CD4-based transmembrane domains, TLR-based transmembrane domains, and combinations thereof.

In some embodiments, the transmembrane domain includes a glycosylphosphatidylinositol (GPI)-anchored domain. In some embodiments, the GPI-anchored domain can be from numerous GPI-anchored proteins. For instance, in some embodiments, the proteins include, without limitation, CD73, CD58, CD59, DAF, CD14, and combinations thereof.

Recruiting Domains

A recruiting domain generally refers to a recombinant protein site that directs the antigen presenting cells towards cancer cells. In some embodiments, the recruiting domain is positioned on the recombinant protein ectodomain.

The recombinant proteins of the present disclosure can include numerous types of recruiting domains. For instance, in some embodiments, the recruiting domains include a receptor. In some embodiments, the receptor directs the antigen presenting cells to cancer cells that secrete a protein that binds to the receptor. In some embodiments, the receptor is CCR6, and the secreted protein is CCL20. In some embodiments, the receptor is an IL-10 receptor, and the secreted protein is IL-10.

Antigen Presenting Cell Activators

Antigen presenting cell activators generally refer to recombinant protein sites that activate or license antigen presenting cells. In some embodiments, the antigen presenting cell activators are positioned on a recombinant protein endodomain.

The antigen present cell activators of the present disclosure can activate antigen presenting cells through numerous mechanisms. For instance, in some embodiments, the antigen presenting cell activators activate the antigen presenting cells through upregulation of the expression of proteins. In some embodiments, the upregulated proteins include, without limitation, major histocompatibility complex proteins, co-stimulatory proteins, pro-inflammatory cytokines, toll-like receptors, or combinations thereof.

The antigen present cell activators of the present disclosure can license antigen presenting cells through numerous mechanisms. For instance, in some embodiments, the antigen presenting cell activators of the present disclosure license antigen presenting cells through upregulation of proteins to efficiently induce a CD8⁺ T cell response. In some embodiments, the upregulated proteins include, without limitation, OX40L, 4-1BBL, and combinations thereof.

The recombinant proteins of the present disclosure can include numerous antigen presenting cell activators. For instance, in some embodiments, the antigen presenting cell activators include, without limitation, CD40, TLR4, TLR2, TLR3, TLR7, TLR8, TLR9, TLR5, FLT3, 4-1BB, LTBR, RANK, and combinations thereof. In some embodiments, the antigen presenting cell activator includes TLR4 and CD40.

In some embodiments, the antigen presenting cell activator includes CD40. In some embodiments, the antigen presenting cells are activated after signal transduction by CD40 to upregulate expression of MHC II, CD80, co-stimulatory protein CD86, pro-inflammatory cytokines, or combinations thereof.

In some embodiments, the antigen presenting cell activator is TLR4. In some embodiments, the antigen presenting cells are activated after signal transduction (e.g., signal transduction by TLR4) to upregulate expression of MHC II, CD80, co-stimulatory protein CD86, pro-inflammatory cytokines, or combinations thereof.

Expression of Recombinant Proteins on Antigen Presenting Cells

Various methods may be utilized to express the recombinant proteins of the present disclosure on antigen presenting cells. For instance, in some embodiments, the expression occurs by introducing a carrier of the present disclosure into the antigen presenting cells. Thereafter, the recombinant protein is expressed by the antigen presenting cells from the nucleotide sequence of the carrier and appended onto a surface of the antigen presenting cells.

The recombinant proteins of the present disclosure can be expressed on antigen presenting cells in various manners. For instance, in some embodiments, the carriers of the present disclosure are introduced into antigen presenting cells in vitro. In some embodiments, the introduction results in the expression of the recombinant proteins of the present disclosure on antigen presenting cells in vitro. Thereafter, the modified antigen presenting cells can be introduced into a subject.

In some embodiments, the carriers of the present disclosure are directly introduced into a subject. In some embodiments, the introduction results in the preferential uptake of the carriers by antigen presenting cells of the subject. In some embodiments, the preferential uptake results in the expression of the recombinant proteins of the present disclosure on antigen presenting cells in vivo.

Various methods may be utilized to introduce the carriers and modified antigen presenting cells of the present disclosure to subjects. For instance, in some embodiments, the introduction occurs by an administration method that includes, without limitation, intravenous administration, subcutaneous administration, transdermal administration, topical administration, intraarterial administration, intrathecal administration, intracranial administration, intraperitoneal administration, intraspinal administration, intranasal administration, intraocular administration, oral administration, intratumor administration, and combinations thereof. In some embodiments, the administering occurs by intravenous administration.

Subjects

The recombinant proteins of the present disclosure may be expressed by antigen presenting cells in various subjects. For instance, in some embodiments, the subject is a human being. In some embodiments, the subject is suffering from cancer. As such, in some embodiments, the methods of the present disclosure can be utilized to treat the cancer in the subject.

Treatment of Cancers

The methods of the present disclosure can be utilized to treat numerous types of cancers in subjects. For instance, in some embodiments, the cancer includes cancers associated with epithelial derived solid tumors, bone marrow-derived tumors, and combinations thereof.

In some embodiments, the cancers to be treated include cancers associated with epithelial derived solid tumors. In some embodiments, such cancers include, without limitation, breast cancer, gastrointestinal carcinomas, head and neck cancer, hepatocellular carcinoma, lung cancer, ovarian cancer, pancreatic cancer, and combinations thereof.

In some embodiments, the cancers to be treated include cancers associated with bone marrow-derived tumors. In some embodiments, such cancers include, without limitation, leukemia, multiple myeloma, and combinations thereof.

The modified antigen presenting cells of the present disclosure can be utilized to treat cancers in subjects through various mechanisms. For instance, in some embodiments, the modified antigen presenting cells of the present disclosure treat cancers in subjects by enabling the immune system to attack the cancer cells of the subject.

In some embodiments, the modified antigen presenting cells of the present disclosure enable T-cells to attack cancer cells by binding to the cancer cells through interaction between the recombinant protein ectodomain of the antigen presenting cells and the antigen of the cancer cells. Thereafter, the interaction results in the presentation of the antigen of the cancer cells on a surface of the antigen presenting cells. Next, the antigen presenting cells present the antigen of the cancer cells on the surface of the antigen presenting cells to the T-cells. As a result, the T-cells become activated to initiate anti-cancer immune responses against the cancer cells.

In some embodiments, the modified antigen presenting cells that activate T-cells include dendritic cells. In some embodiments, the modified antigen presenting cells that activate T-cells include macrophages. In some embodiments, the macrophages bind to the cancer cells through interaction between the recombinant protein ectodomain of the macrophages and the antigen of the cancer cells to result in the killing of the cancer cells by the macrophages. In some embodiments, the killing of the cancer cells by the macrophages occurs by phagocytosis of the cancer cells by the macrophages.

Carriers

Carriers generally refer to delivery vectors that are able to express the recombinant proteins of the present disclosure in antigen presenting cells and thereby form the modified antigen presenting cells of the present disclosure. The carriers of the present disclosure generally include a nucleotide sequence that expresses the recombinant proteins of the present disclosure.

The carriers of the present disclosure can be in various forms. For instance, in some embodiments, the carriers of the present disclosure include, without limitation, particles, microparticles, nanoparticles, micelles, lentiviruses, retroviruses, and combinations thereof. In some embodiments, the carriers of the present disclosure are in the form of nanoparticles.

The nucleotide sequences in the carriers of the present disclosure can also be in various forms. For instance, in some embodiments, the nucleotide sequence includes, without limitation, DNA, mRNA, and combinations thereof. In some embodiments, the nucleotide sequence includes a DNA on a plasmid.

In some embodiments, the nucleotide sequences of the present disclosure may be encapsulated within the carriers of the present disclosure. For instance, in some embodiments, the nucleotide sequences of the present disclosure may be encapsulated within particles. In some embodiments, the nucleotide sequences may also be embedded with polymers.

Applications and Advantages

The methods and modified antigen presenting cells of the present disclosure provide numerous advantages. For instance, in some embodiments, the modified antigen presenting cells of the present disclosure are expected to provide minimal side effects when introduced to subjects. For instance, in some embodiments where there is no mutated gene in normal tissue to trigger specific immune responses, the off-target side effects are negligible (i.e., binding of modified antigen presenting cells to normal cells will not trigger a specific immune response to kill normal cells). Moreover, since the modified antigen presenting cells of the present disclosure can be activated through interaction with cancer cells, no additional adjuvants are necessary to provide such activation.

Furthermore, in some embodiments, the methods and carriers of the present disclosure can be utilized to preferentially express the recombinant proteins of the present disclosure on the antigen presenting cells of subjects in vivo, thereby eliminating the need for harvesting and modifying the antigen presenting cells in vitro. As such, the modified antigen presenting cells and methods of the present disclosure can be used to treat numerous types of cancers in various subjects in a cost effective and efficient manner.

Moreover, in some embodiments, after binding to cancer cells, the modified antigen presenting cells of the present disclosure (e.g., GPI anchored scFv modified DCs or CAR DCs) could uptake antigens from cancer cells to readily trigger epitope spreading. Therefore, in such embodiments, there is no need to identify highly specific antigens for each patient. A general antigen to guide the modified antigen presenting cells of the present disclosure (e.g., GPI anchored scFv modified DCs or CAR DCs) to cancer sites to start this process can be sufficient. For instance, in some embodiments, the EPCAM protein on epithelial derived solid tumors is a sufficient target.

### Additional Embodiments

Reference will now be made to more specific embodiments of the present disclosure and experimental results that provide support for such embodiments. However, Applicants note that the disclosure below is for illustrative purposes only and is not intended to limit the scope of the claimed subject matter in any way.

### Example 1. Remodeled Dendritic Cells by GPI-Anchored scFv or Chimeric Antigen Receptors (CAR)

This Example demonstrates the expression of a single chain variable fragment (scFv) on dendritic cell (DC) membranes either through GPI anchors (modified cells referred to herein as “GPI anchored scFv modified DC”) or chimeric antigen receptors (modified cells referred to herein as “CAR DC”). The dendritic cells efficiently target and bind to cancer cells. Thereafter, the dendritic cells get fully activated through the binding. The fully activated dendritic cells can uptake and present cancer specific antigens, express co-stimulatory molecules and produce pro-inflammatory cytokines to activate the immune system.

Numerous differences exist between CAR DC and GPI anchored scFv modified DC. For instance, there are some intracellular domains in CAR DC structures that are absent from GPI anchored scFv modified DC. After binding with cancer cells, these intracellular domains could fully activate dendritic cells. On the other hand, in order to activate GPI anchored scFv modified DC, the cells must be treated with adjuvants, such as LPS or CpG. 2.

Moreover, strategies used to translocate fusion proteins to cell membranes are different in CAR DC and GPI anchored scFv modified DC. For instance, CD8 transmembrane domains are utilized for CAR DC while a GPI anchor is used for GPI anchored scFv modified DC.

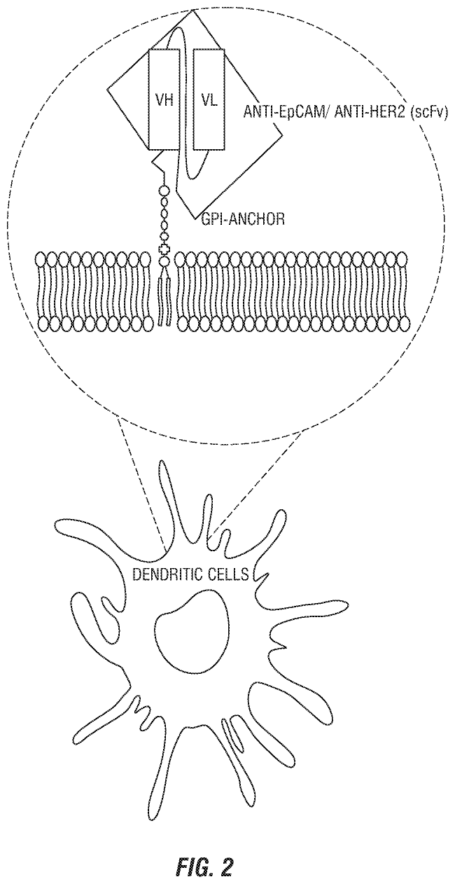

FIG. 2 provides an illustration of a GPI anchored scFv modified DC. A single-chain variable fragment (scFv) of an antibody was expressed on the dendritic cell (DC) surface so that DC could recognize and bind with particular cancer cells. By way of background, scFv is a fusion protein of the variable regions of the heavy chain (VH) and light chain (VL) from an antibody (anti-EpCAM or anti-Her2 as examples here). VH and VL are connected by a linker region to increase the flexibility. The scFv is capable of recognizing and binding with particular molecules (EpCAM or Her2) on target cell surfaces.

To translocate the scFv to the surface of DC, Applicant fused scFv with an N-terminus secretion signal and a c-terminus Glycosylphosphatidylinositol (GPI) anchor. These two domains help the fusion protein to be recognized and sorted to the endoplasmic reticulum (ER) and finally anchor to the cell membrane through a secretory pathway.

Dendritic cells expressing this kind of GPI anchored scFv could bind with particular cancer cells, uptake antigen from them, and then activate specific immune responses against them. As such, the aforementioned dendritic cells provide an optimal platform and could be utilized to apply numerous scFvs to recognize numerous molecules on cancer cell surfaces. In order to establish preliminary data for the aforementioned platform, Applicant utilized anti-EpCAM GPI (aEpCAM-GPI) or anti-Her2 GPI (aHer2-GPI).

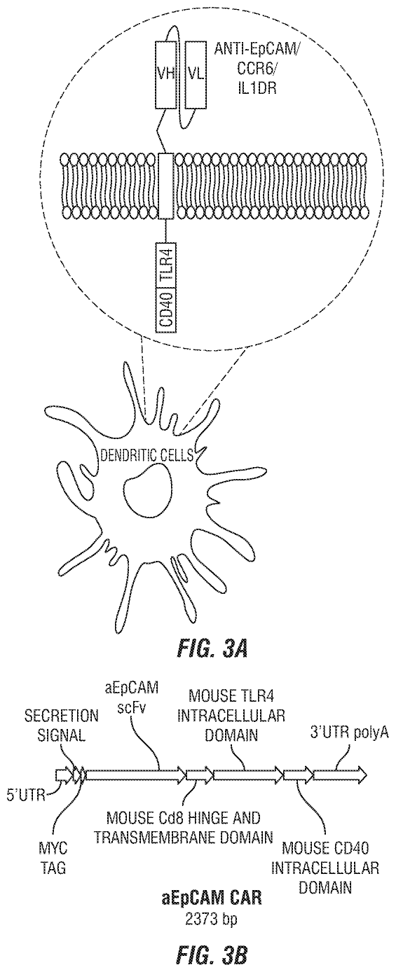

FIG. 3 provides a structure of Applicant's CAR DC. There are several domains in this structure playing different functions. As a consequence, this structure encodes a fusion protein on dendritic cell membranes, thereby helping CAR DCs recognize cancer cells, uptake antigen and be fully activated to trigger a specific immune response. In this Example, the extracellular domain is a single-chain variable fragment (scFv), targeting EpCAM (same sequence as used in “GPI anchored scFv remodeling dendritic cells”, which is human EpCAM specific) on cancer cell surfaces. CCR6 or IL10R can also be utilized to target cancer microenvironments. For the preliminary data, Applicant utilized scFv targeting EpCAM (aEpCAM) only.

In the example illustrated in FIG. 3, Applicant utilized the CD8 transmembrane domain (combined with the secretion signal in N-terminal) to translocate the CAR DCs to the cell membrane. The CD8 hinge domain makes aEpCAM scFv more flexible, which could enhance the efficiency.

For the intracellular domain, Applicant utilized TLR4 and CD40 intracellular domains in tandem. TLR4 is the receptor of LPS (lipopolysaccharide), the most efficient dendritic cell agonist. CD40 signaling is desirable for dendritic cell licensing. Applicant believes that the binding of dendritic cells to cancer cells through aEpCAM scFv could activate TLR4 and CD40 signaling pathways, which could fully activate and license dendritic cells, thereby making them ready to activate the immune system.

In the N-terminal, Applicant added a Myc tag to detect the expression of CAR. In addition, the 5′UTR and 3′UTR+poly A were utilized to enhance the expression efficacy of CAR.

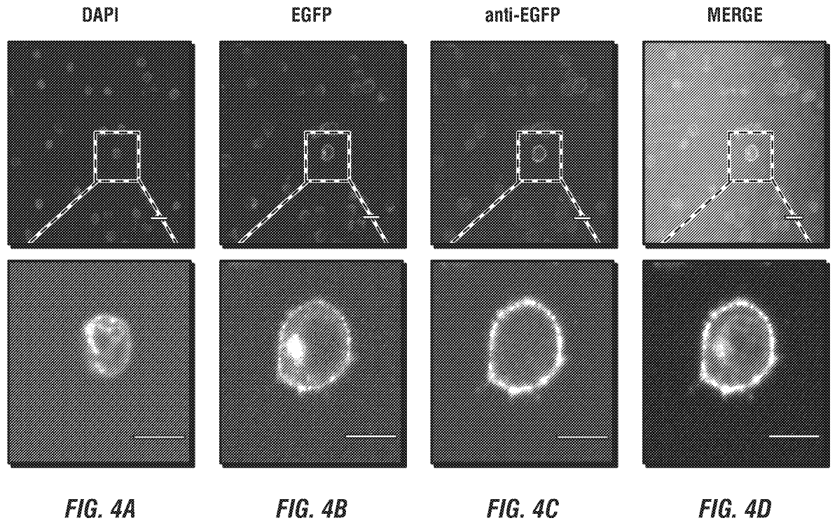

FIG. 4 provides experimental results establishing that the GPI anchors of GPI anchored scFv modified DCs could translocate the fusion protein to cell surfaces. The experimental results confirm that the secretion signal and GPI anchor could successfully translocate the fusion protein to the cell membrane.

For the experiments in FIG. 4, Applicant utilized a reporter fusion protein (fluorescence protein EGFP fused with secretion signal and GPI anchor) to prove the aforementioned hypothesis. By immunofluorescence, Applicant detected both signal from EGFP and fluorescence labeled anti-EGFP antibody. As shown by the images in FIG. 4, Applicant can see clearly that the fusion protein is located on cell membranes.

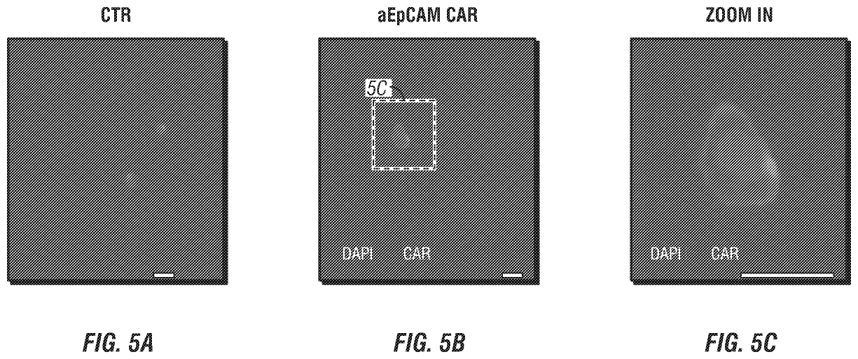

FIG. 5 provides experimental results that further affirm that Applicant's CAR DC is located on cell membranes. In particular, the experimental results summarized in FIG. 5 affirm that the secretion signal and CD8 transmembrane domain could successfully translocate Applicant's CAR to the cell membrane. In the aforementioned experiments, Applicant used fluorescence labeled anti-myc tag antibody to detect the subcellular location of the CAR.

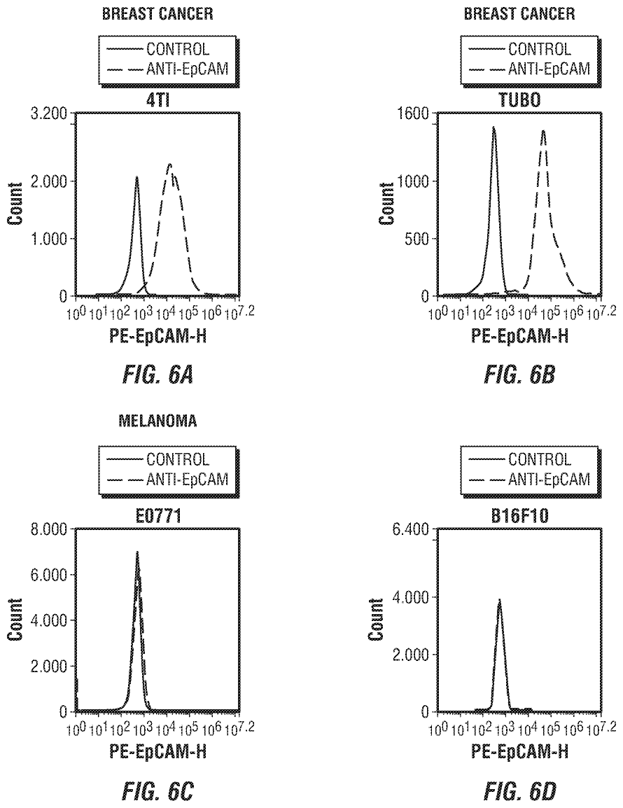

FIG. 6 provides experiments demonstrating the specificity of aEpCAM scFv. In particular, Applicant detected the EpCAM expression of murine cell lines. Applicant observed that 4T1 and TUB 0 are EpCAM positive while B16F10 and E0771 cells are EpCAM negative.

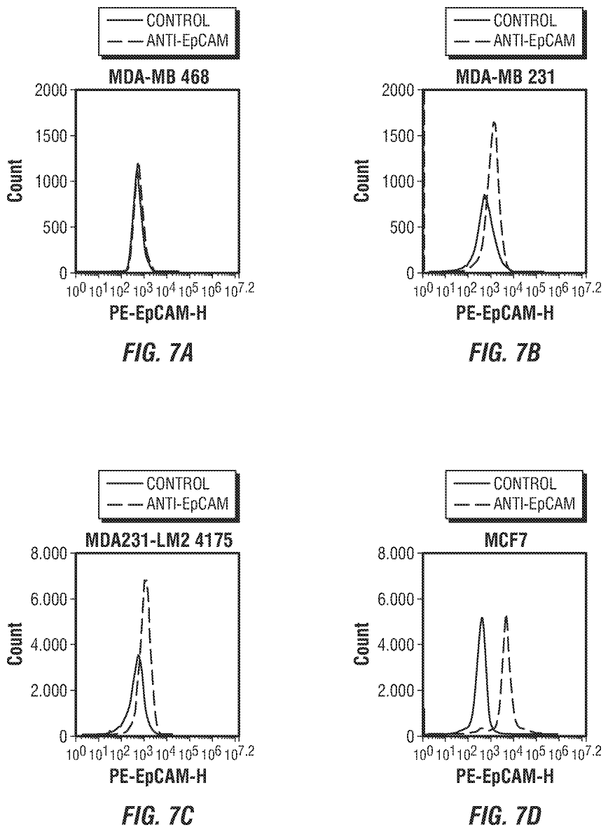

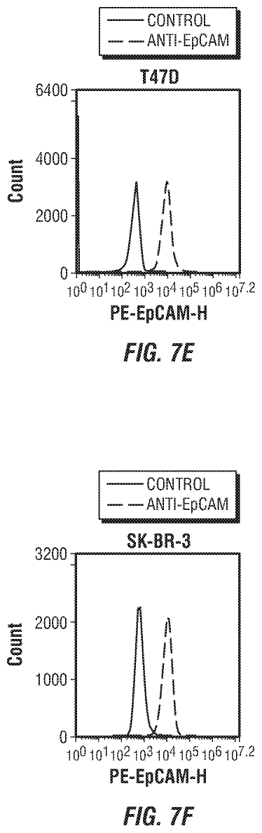

FIG. 7 provides additional experiments that demonstrate the specificity of aEpCAM scFv. The human cancer cell lines MCF7, T47D and SK-BR-3 have over-expressed EpCAM, while MDA-MB 231 and MDA231-LM2 4175 have medium levels of EpCAM. MDA-MB 468 is EpCAM negative.

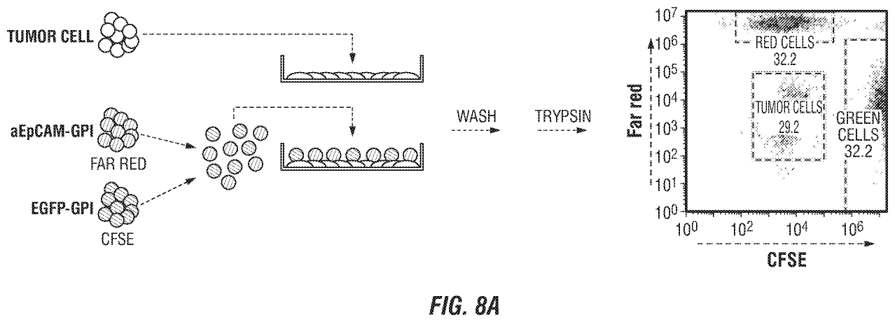

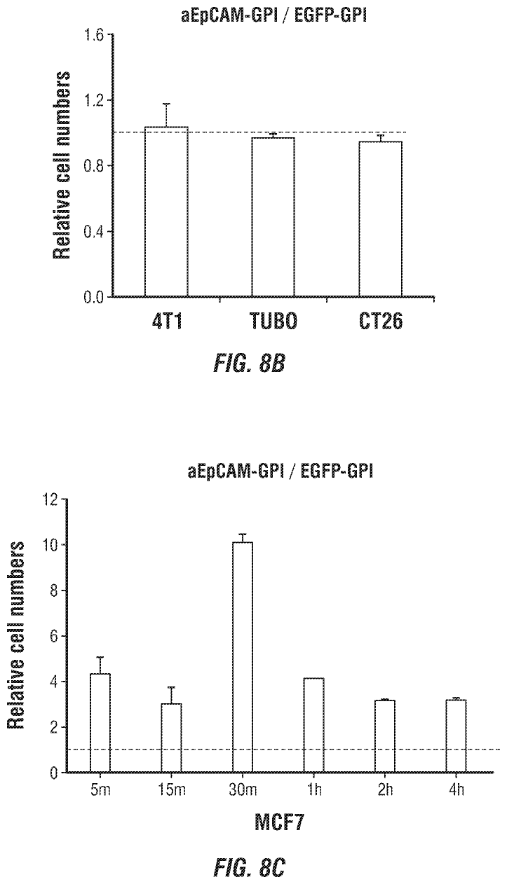

FIG. 8 provides additional experimental results that demonstrate the specificity of aEpCAM scFv. In particular, FIG. 8 summarizes a cell-cell interaction assay to test the specificity of Applicant's aEpCAM scFv. Applicant expressed aEpCAM-GPI or EGFP-GPI (as a control) on dendritic cell membranes and then labeled aEpCAM-GPI or EGFP-GPI cells with different dyes (Far red or CFSE). The cells were then mixed together at a ratio of 1:1 and co-cultured with different cancer cell lines. After an indicated time, the unbound cells were extensively washed away and the bound cells were collected and analyzed by flow cytometry.

FIG. 8 shows the relative binding cell numbers (aEpCAM-GPI/EGFP-GPI). The values above 1 means dendritic cells have stronger ability to bind cancer cells compared to EGFP-GPI expressing control. The experimental results demonstrate that Applicant's aEpCAM scFv is human specific since they could not recognize murine EpCAM. Moreover, aEpCAM-GPI increases the interaction with human cell line MCF7 but not murine cells 4T1, TUBO and CT26.

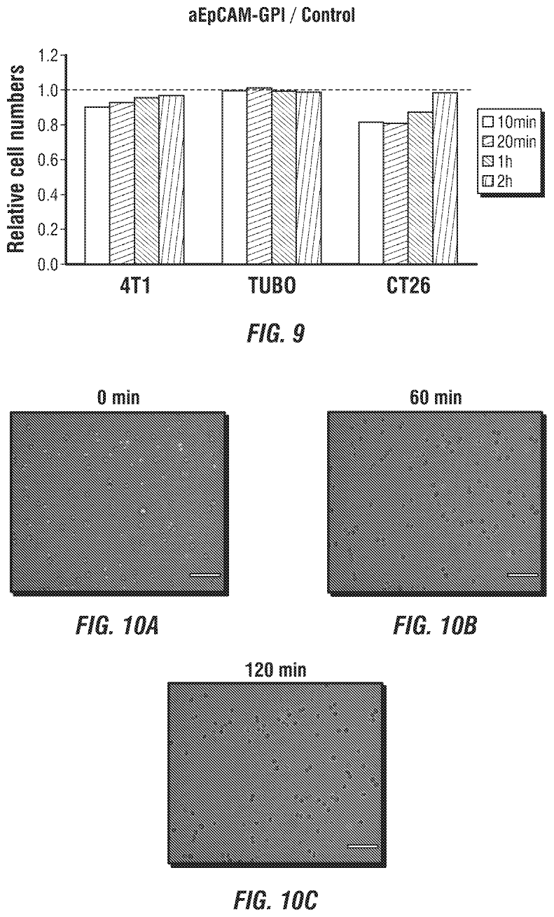

FIG. 9 demonstrates that the binding ability of CMV promoter (cytomegalovirus promoter, driving gene expression) driven aEpCAM-GPI expressing DC to cancer cells does not change. In particular, FIG. 9 summarizes results from a cell-cell interaction assay. DC2.4 expressing CMV driven aEpCAM-GPI does not show enhanced binding ability to both murine (4T1 and TUBO) and human (MCF7 DR) cancer cell lines compared to control cell lines (DC2.4 transfected with an empty vector). Since the CMV promoter does not work in this system, Applicant utilized an EF1a promoter to drive aEpCAM-GPI or EGFP-GPI in all other experiments.

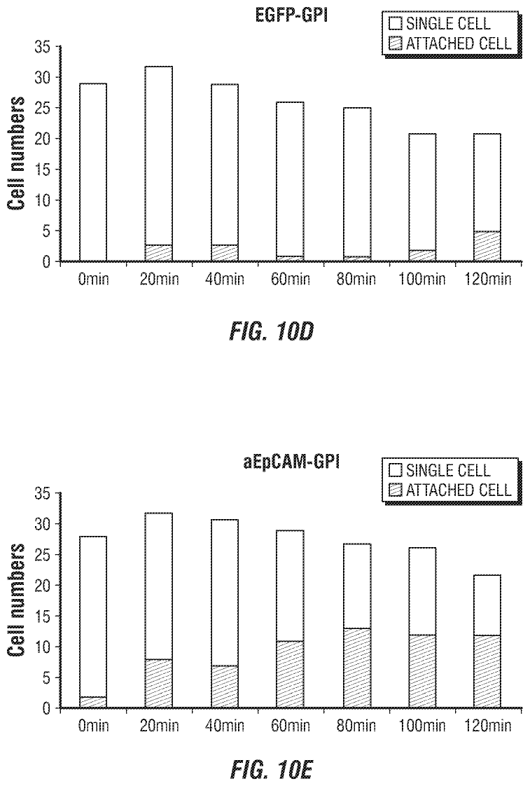

FIG. 10 provides additional experimental results that demonstrate the specificity of aEpCAM scFv. In particular, the experimental results demonstrate that the aEpCAM-GPI expressing DC has increased ability to interact with human EpCAM (hEpCAM) positive cancer cells. Applicant labeled aEpCAM-GPI (red) or EGFP-GPI (green) cells with different dyes. Thereafter, Applicant mixed the cells together with MCF7 (not labeled) at a ratio of 1:1:2 and monitored cell-cell interaction by time lapse imaging. Applicant took photos at different time points and counted the single unattached cells and the cells binding with MCF7. The results demonstrate that there are more aEpCAM-GPI cells attaching to MCF7. The results also demonstrate that aEpCAM-GPI help DC to recognize and bind hEpCAM positive cells.

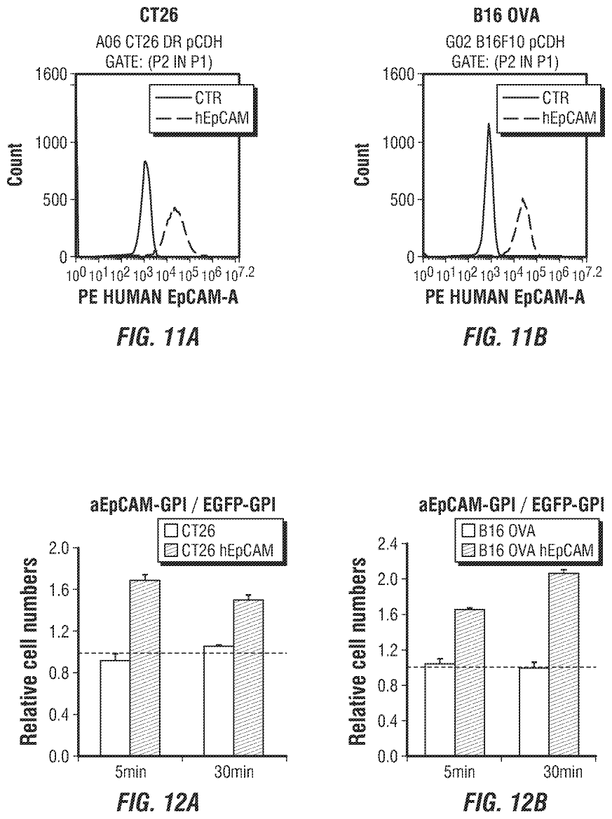

FIG. 11 provides additional experimental results that demonstrate the specificity of aEpCAM scFv. Since Applicant's aEpCAM scFv is human specific, Applicant expressed human EpCAM (hEpCAM) in murine cancer cell lines. The results in FIG. 11 represent the overexpression efficiency detected by flow using a human EpCAM specific antibody. In such, the results in FIG. 11 indicate that hEpCAM is force expressed in murine cancer cell lines.

FIG. 12 provides additional experimental results that demonstrate the specificity of aEpCAM scFv. To test the cancer models, Applicant performed cell-cell interaction assays using hEpCAM expressing CT26 and B16 OVA murine cell lines. The results demonstrate that, after expression of hEpCAM, the murine cancer cells could be efficiently recognized by Applicant's aEpCAM-GPI expressing dendritic cells.

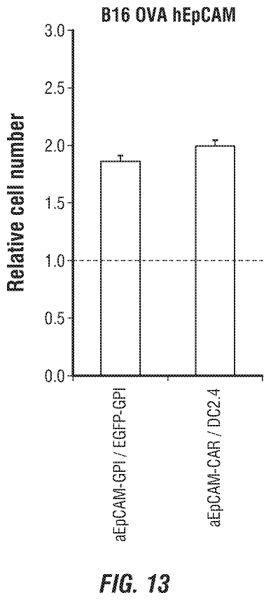

FIG. 13 shows experimental results demonstrating that CAR expression enhances the binding ability of DCs to human EpCAM positive cancer cells. Since the extracellular domain (anti-EpCAM scFv) is the same as the one used in GPI anchored scFv remodeled dendritic cells, which is human EpCAM specific, Applicant performed cell-cell interaction assays using hEpCAM expressing murine cell lines to test the binding ability of Applicant's CAR. After expression of hEpCAM, the murine cancer cells could be efficiently recognized by Applicant's CAR expressing dendritic cells to the similar extent as aEpCAM-GPI. The results demonstrate that CAR expressing DCs recognize hEpCAM positive cancer cells.

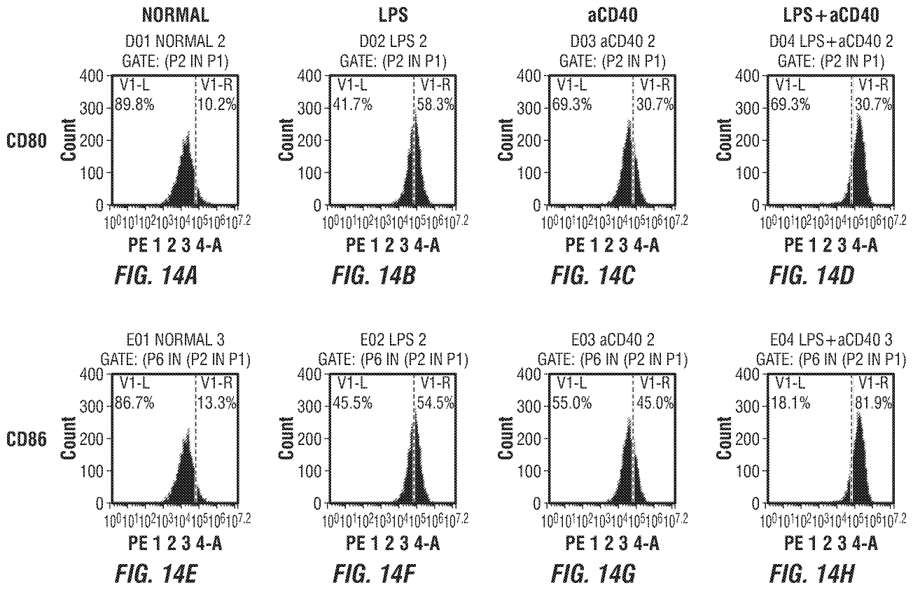

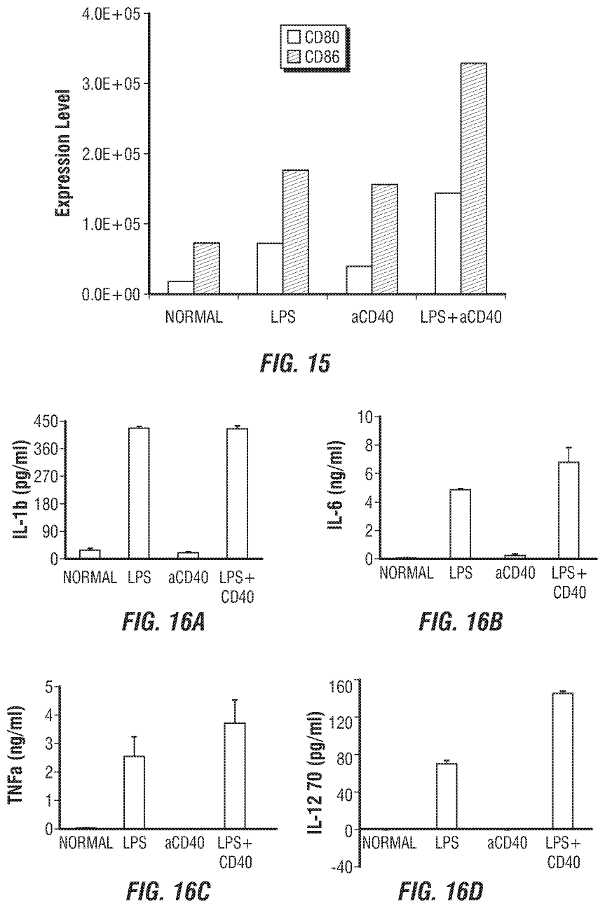

FIGS. 14 and 15 show experimental results demonstrating that TLR4 and CD40 have synergistic effects. To demonstrate the rationality to apply TLR4 and CD40 intracellular domains in tandem, Applicant first tested whether TLR4 and CD40 signaling pathways have synergistic effects. LPS is the ligand of TLR4. The anti-CD40 (aCD40) antibody is a commercial agonist of CD40. CD80 and CD86 are commonly used biomarkers for dendritic cell activation. Applicant used LPS, aCD40 or LPS+aCD40 to treat bone-marrow derived dendritic cells (BMDC) and analyzed CD80 and CD86 expression by flow cytometry. The flow cytometry results are shown in FIG. 14. The quantification of the flow cytometry results are shown in FIG. 15. The results demonstrate that TLR4 and CD40 pathways have synergistic effects on the activation of dendritic cells.

FIG. 16 provides additional experimental results that further demonstrate that TLR4 and CD40 have synergistic effects in activating dendritic cells. In particular, other than biomarkers, activated dendritic cells resulted in the enhanced production of numerous pro-inflammatory cytokines, including the following downstream cytokines of TLR4 and CD40 pathways: IL-1b (FIG. 16A), IL-6 (FIG. 16B), TNFα (FIG. 16C) and IL-12p70 (FIG. 16D). In particular, the results establish that the combined treatment of dendritic cells with TLR4 and CD40 agonists resulted in the enhanced production of cytokines.

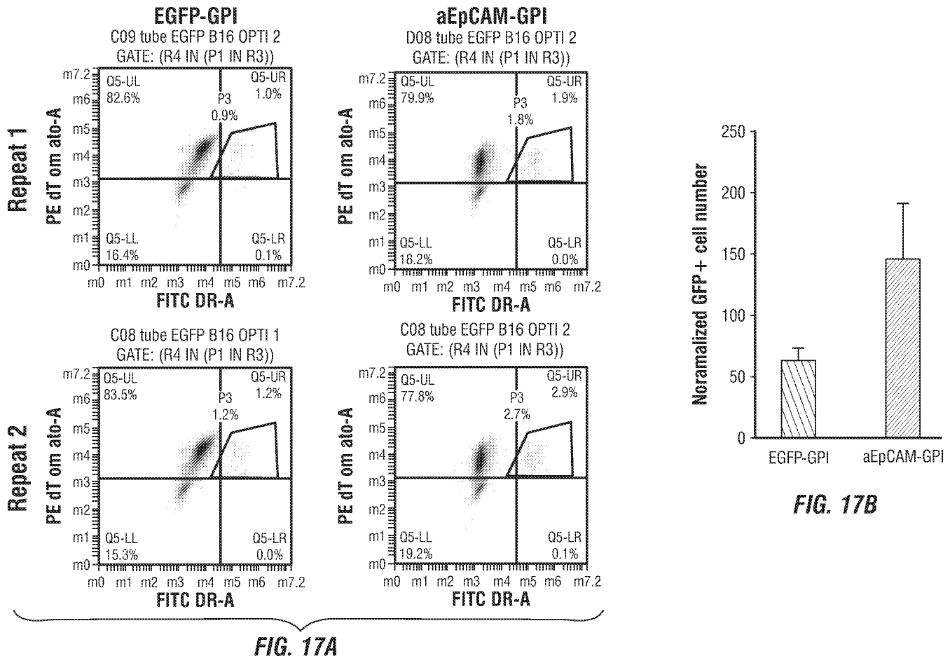

FIG. 17 provides experimental results from a dendritic cell antigen uptake assay. To mimic a dendritic cell antigen uptake process, Applicant labeled B16 OVA hEpCAM cell lines with EGFP (EGFP-GPI does not affect this assay since EGFP-GPI is on the membrane and the intensity is very low compared to those EGFPs from cancer cells). Applicant co-cultured EGFP labeled B16 OVA hEpCAM with aEpCAM-GPI or EGFP-GPI expressing DCs. After a few hours, Applicant used flow cytometry (FIG. 17A) to detect EGFP positive (uptaking EGFP from cancer cell) DCs. The results indicates that there are more EGFP positive cells in aEpCAM-GPI DC after co-culture, which means aEpCAM-GPI expressing DC has increased ability to uptake antigen from cancer cells (FIG. 17B).

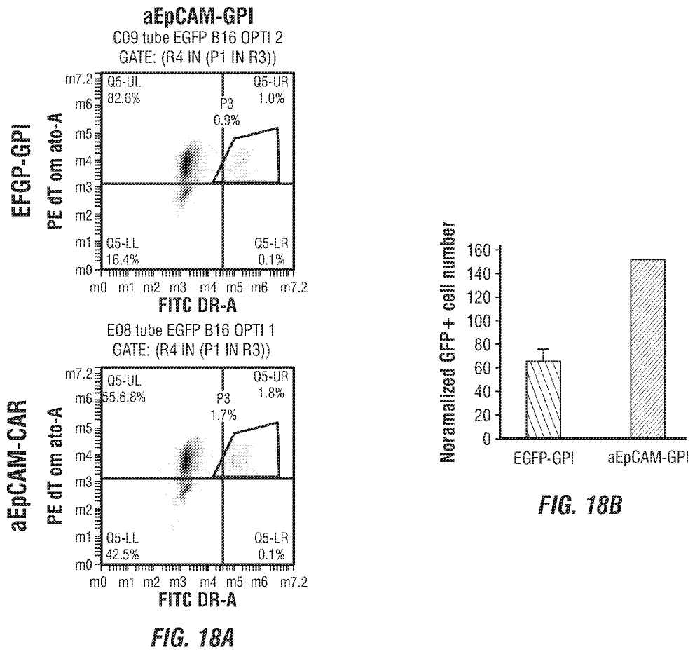

FIG. 18 provides experimental results demonstrating that CAR-DC has more anti-tumor potential. Dendritic cells can uptake cancer-specific antigen from debris, exosomes or apoptotic cancer cells to trigger anti-tumor immune responses. Since Applicant's CAR-DC could recognize and bind to cancer cells, they may have more potential to uptake antigens. Applicant expressed EGFP in hEpCAM expressing cancer cells and co-cultured them with CAR-DC. If dendritic cells uptake antigen (EGFP as a reporter) from cancer cells, they should be EGFP positive. Applicant analyzed the percentage of EGFP positive dendritic cells by flow cytometry. The results indicate that CAR-DCs have a stronger ability to uptake antigens from cancer cells.

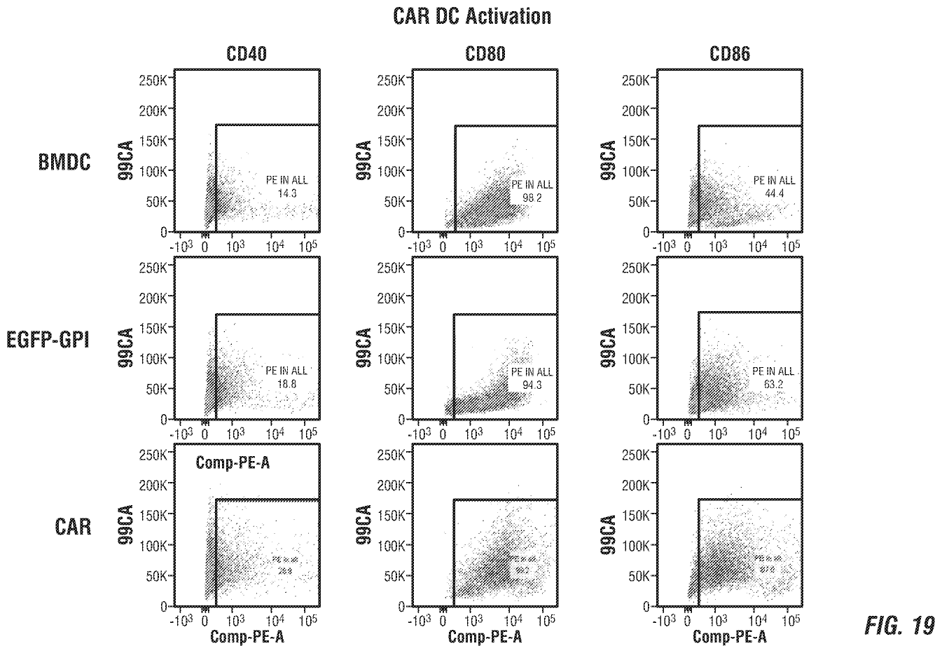

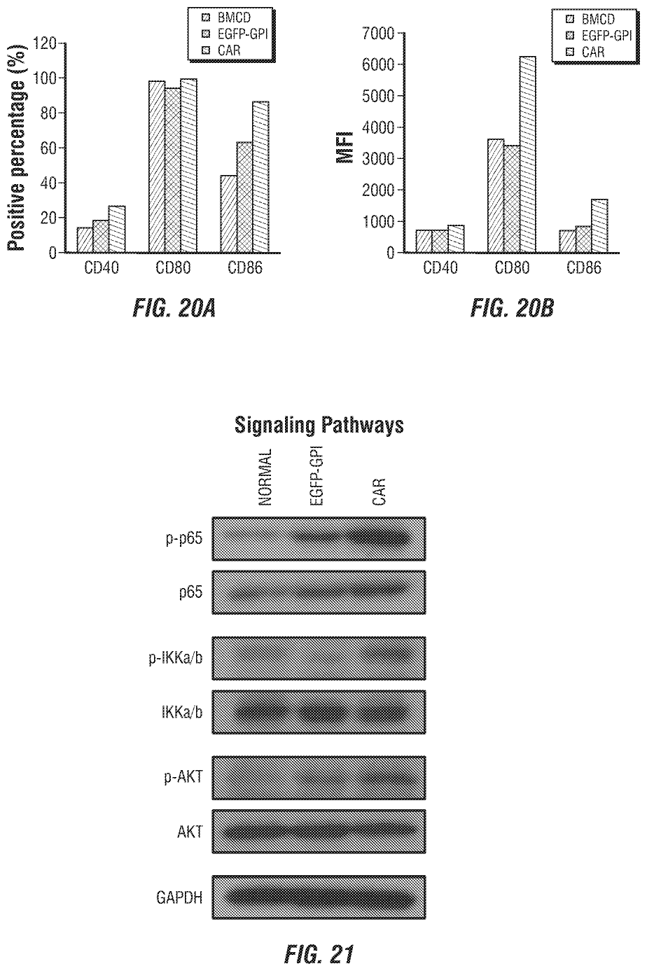

FIGS. 19 and 20 provide additional experimental results demonstrating that CAR-DC has more anti-tumor potential. Since Applicant's CAR has TLR4 and CD40 intracellular domains, it should be able to activate dendritic cells after expression. In particular, CD8 transmembrane domain can trigger dimerization of CAR. Applicant used flow cytometry (FIG. 19) to measure activation associated biomarkers (CD40, CD80 and CD86) expressions. The normal BMDC and BMDC expressing EGFP-GPI were used as controls. The flow cytometry results were also quantified, where both positive percentage (%) and intensity (MFI) are shown (FIG. 20). The results affirm that CAR expression can activate dendritic cells.

To further affirm that the CAR TLR4 and CD40 intracellular domains can activate dendritic cells, Western blot analyses were performed on the cells after CAR expression to test for activation of downstream signaling pathways. The results, which are summarized in FIG. 21, show that CAR expression can activate NF-kappa-B (p-p65 and p-IKKa/b level is increased) and AKT signaling (p-AKT level is increased). As such, the result further affirm that CAR expression can activate dendritic cells.

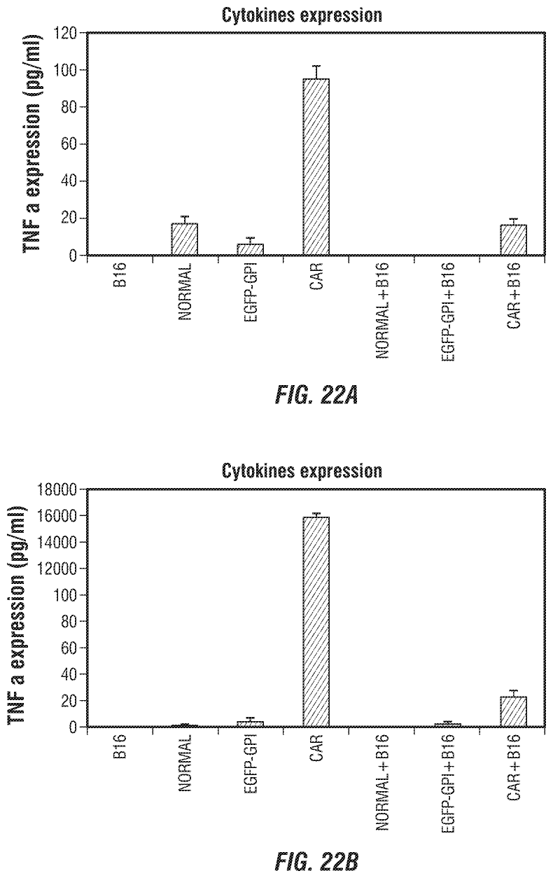

In order to fully activate an immune response, dendritic cells should preferably secrete many cytokines, including TNFa and IL-6. To further affirm the activation of dendritic cells, Applicant co-cultured normal BMDC, BMDC expressing EGFP-GPI or CAR with cancer cells (B16 expressing hEpCAM) and measured TNFa and IL-6 expression. The results, which are summarized in FIG. 22, indicate that CAR expressing dendritic cells are more potent to secrete TNFa and IL-6.

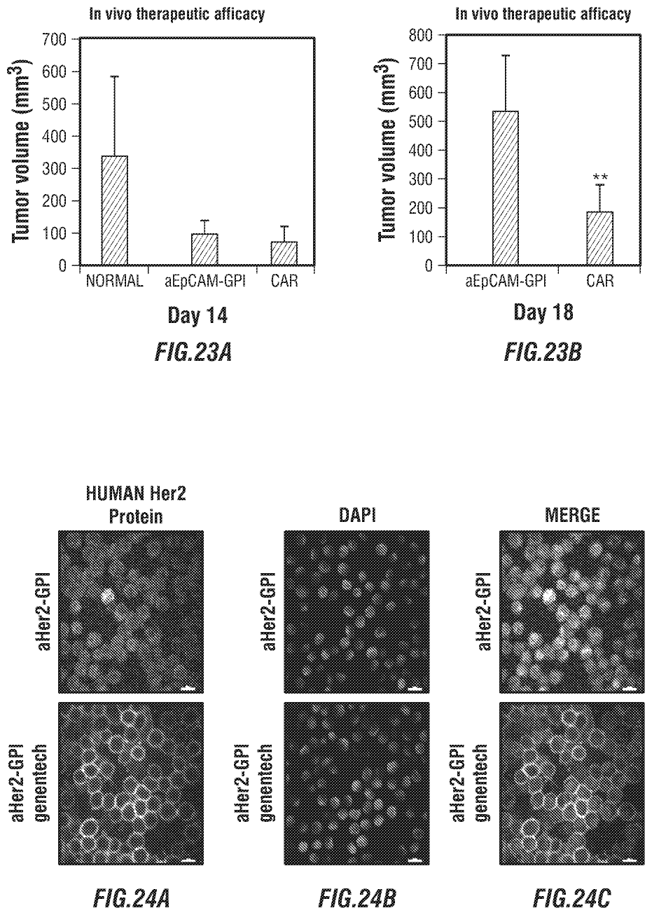

In order to test the therapeutic efficacy of aEpCAM-GPI and CAR DC in vivo, Applicant used aEpCAM-GPI or CAR BMDC to treat a B16-OVA-hEpCAM tumor model. The results of this assay are shown in FIG. 23. Compared to normal BMDC, both aEpCAM-GPI and CAR BMDC inhibited tumor growth significantly. The results indicate that both aEpCAM-GPI and CAR DC are able to inhibit tumor growth, while CAR DC was more effective.

### Example 2. Remodeled Dendritic Cells by GPI-Anchored Anti-Her2 scFv

In Example 1, Applicant demonstrated that dendritic cells with GPI-anchored anti-EpCAM single chain variable fragments (scFv) had anti-tumor effects. In this Example, Applicant demonstrates that that dendritic cells with GPI-anchored anti-Her2 single chain variable fragments (scFv) also have anti-tumor effects.

FIG. 24 provides images illustrating that αHer2-GPI is located on cell membranes and is able to bind human Her2 proteins. Applicant labeled human Her2 proteins with a fluorescent dye and co-cultured the cells with αHer2-GPI expressing dendritic cells. For this experiment, Applicant utilized two different sequences of αHer2: (1) αHer2-GPI; and αHer2-GPI Genentech. Only αHer2-GPI Genentech could recognize the human Her2 protein. In sum, the results in FIG. 24 illustrate that αHer2-GPI Genentech is located on cell membranes and could recognize the human Her2 protein.

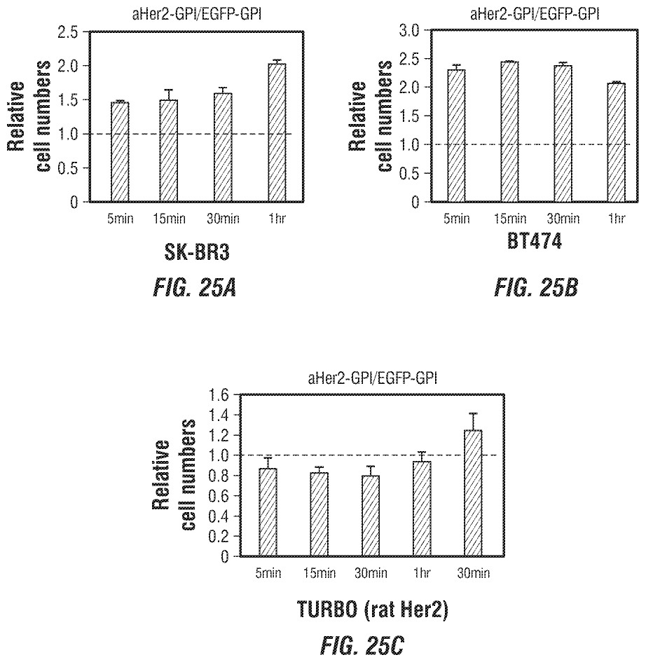

FIG. 25 provides data illustrating that αHer2-GPI could only recognize human Her2. In particular, FIG. 25 provides results derived from a cell-cell interaction assay, demonstrating that dendritic cells expressing αHer2-GPI have enhanced ability to interact with Her2 positive human cell lines (SK-BR3 and BT474) but not rat Her2 positive TUBO cells. As such, αHer2-GPI is human specific.

### Example 3. pDNA Delivery Nanoparticles

In this Example, Applicant provides data related to plasmid DNA (pDNA) delivery nanoparticles for delivering plasmids expressing aEpCAM-GPI or aEpCAM CAR fusion genes to dendritic cells both in vitro and in vivo. Also provided are measurements related to sizes, zeta potentials, encapsulating capacities and expression efficacies of the naoparticles in the HEK293FT cell line and the dendritic cell line DC2.4.

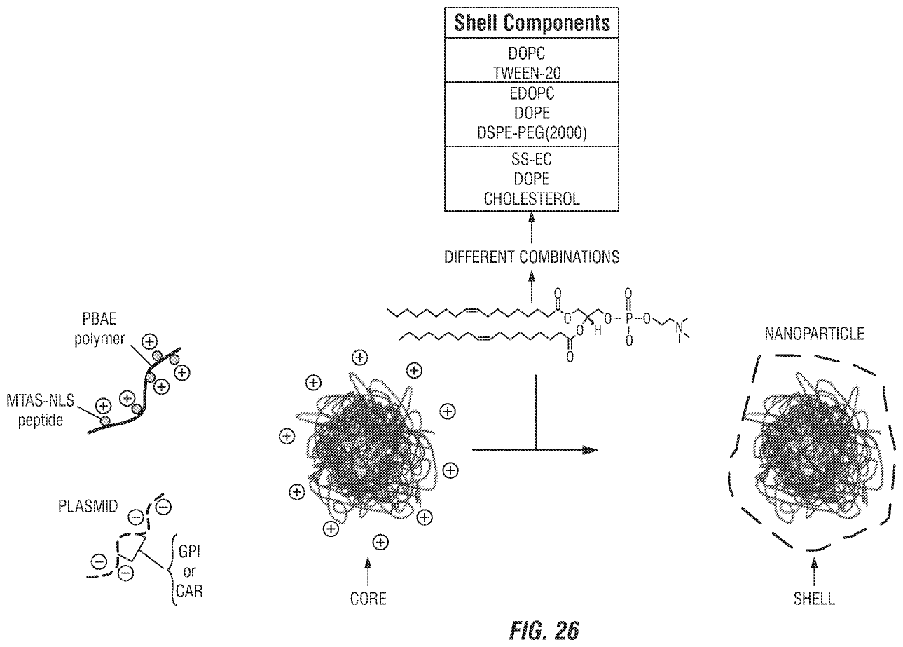

FIG. 26 provides depictions of the nanoparticles in this Example. The nanoparticles are core-shell liposomes. The positively charged peptides attach to a polymer and integrate with negatively charged DNA to form a condensed core. The core is encapsulated by a lipid shell, which consists of different components to mimic the membrane of cell and facilitate the uptake by dendritic cells.

For the core, Applicant utilized a PBAE (poly(beta-amino ester)) polymer conjugated with MTAS-NLS (microtubule-associated sequences (MTAS) and nuclear localization signals (NLS)), a peptide guiding pDNA to the nucleus of target cells. This positive charged complex integrates with negative charged plasmids to form a condensed core.

For the shell structure, Applicant has three different combinations as listed in FIG. 26. DOPC represents 1,2-dioleoyl-sn-glycero-3-phosphocholine, a naturally occurring phospholipid. TWEEN 20 is an emulsifying agent. EDOPC is 1,2-dioleoyl-sn-glycero-3-ethylphosphocholine (chloride salt). O-alkyl phosphatidylcholines constitute the first chemically stable triesters of biological lipid structures and the first cationic derivatives of phospholipids consisting entirely of biological metabolites linked with ester bonds. The lipid has low toxicity and is biodegradable. DOPE is 1,2-dioleoyl-sn-glycero-3-phosphoethanolamine. DSPE-PEG(2000) is 1,2-distearoyl-sn-glycero-3-phosphoethanolamine-N-[amino(polyethylene glycol)-2000] (ammonium salt), a component that provides extra steric stability to the particles. SS-EC is a commercial SS-cleavable and pH-responsive lipid-like material. This material contains dual sensing motifs that can respond to the intracellular environment. Tertiary amines respond to an acidic compartment (endosome/lysosome) for membrane destabilization, and a disulfide bond that can cleave in reductive environments (cytoplasm). These two units synergize for efficient intracellular delivery of encapsulated drugs with enhanced biosafety. Cholesterol is utilized to make the shell of the nanoparticles firm.

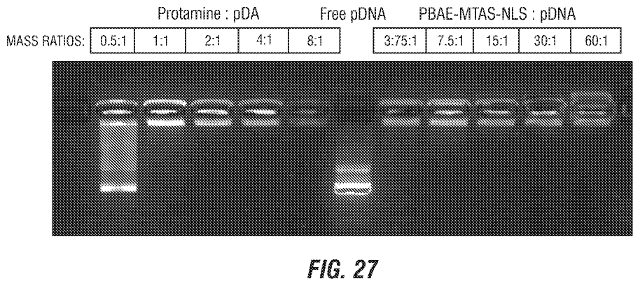

FIG. 27 provides agarose gel data regarding the ability of PBAE-MTAS-NLS to trap pDNA. Free pDNA detected as a sole band is used as a control. The trapped pDNA (forming a core with peptide) could not run into the gel since the core is too large and positively charged. As shown in FIG. 27, PBAE-MTAS-NLS could efficiently trap pDNA, as no free plasmid is left. Different mass ratios are shown. The 15:1 is used for all further experiments. Protamine is another commonly used protein to form core with DNA. Applicant's data confirm the ability of PBAE-MTAS-NLS to trap DNA (except the ratio of 0.5:1). However, in further studies, Applicant did not observe any gene expression from protamine particles (data not shown here). In sum, the results indicate that PBAE-MTAS-NLS can trap pDNA.

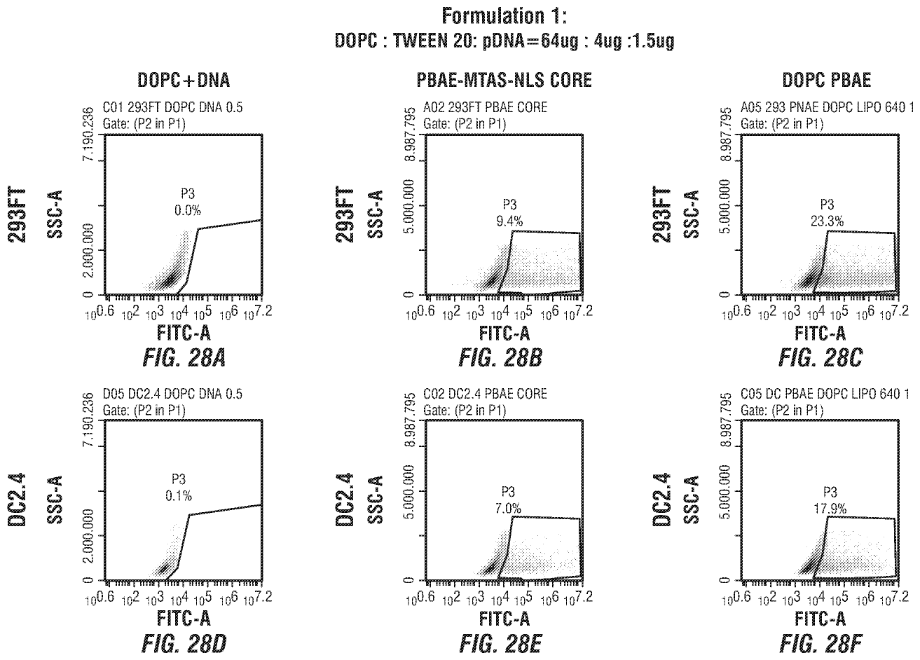

FIG. 28 provides data indicating that both core and shell are necessary in pDNA nanoparticles for intensive gene expression. A formulation that included DOPC and TWEEN 20 was utilized as an example. Without the core (DOPC+DNA only), no gene expression was detected. The plasmid encoding EGFP, a fluorescent protein, was detected by flow cytometry.

The core only (the PBAE-MTAS-NLS core) provided a modest gene expression. However, the core and shell (DOPC PBAE) triggered an intensive gene expression. The results were confirmed in both HEK293FT cells and DC2.4 cells. The size of core and shell particle is around 600 nm, and the zeta potential is around 15 mV. In sum, the results indicate that both the core and shell are necessary for intensive gene expression.

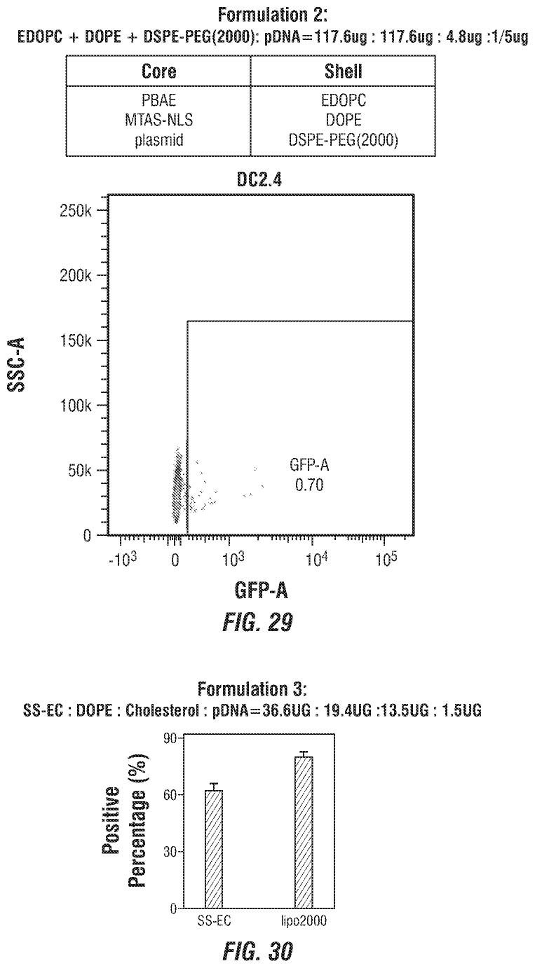

FIG. 29 provides data illustrating that gene expression in dendritic cell lines is possible from a pDNA delivery nanoparticle containing “formulation 2.” Similarly, FIG. 30 provides data illustrating that gene expression in 293FT cell lines is possible from a pDNA delivery nanoparticle containing “formulation 3.” The size of the particle with PBAE and MTAS-NLS in FIG. 30 is around 122 nm, and the zeta potential is around 35 mV.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A method of expressing a recombinant protein on antigen presenting cells, said method comprising:
introducing a carrier into the antigen presenting cells,
wherein the carrier comprises a nucleotide sequence expressing the recombinant protein,
wherein the recombinant protein is expressed by the antigen presenting cells from the nucleotide sequence and appended onto a surface of the antigen presenting cells, and
wherein the recombinant protein comprises:
an ectodomain that is positioned on the surface of the antigen presenting cells, wherein the ectodomain is capable of preferentially binding to an antigen of cancer cells, and
a transmembrane domain comprising at least one region embedded in the antigen presenting cell membrane,
wherein the transmembrane domain comprises a glycosylphosphatidylinositol (GPI)-anchored domain, and
wherein the transmembrane domain serves as an anchor for maintaining the ectodomain of the recombinant protein on the surface of the antigen presenting cells.

2. The method of claim 1, wherein the recombinant protein ectodomain is selected from the group consisting of whole antibodies, single chain antibodies, nanobodies, aptamers, antibody fragments, portions of antibodies, scFV portions of antibodies, peptides, and combinations thereof.

3. (canceled)

4. The method of claim 1, wherein the cancer cell antigen is selected from the group consisting of moieties, proteins, glycoproteins, EGF receptors, MUC-1, lipids, EPCAM, HER-2 receptors, and combinations thereof.

5. The method of claim 1, wherein the cancer cell antigen is an EPCAM protein, and wherein the recombinant protein ectodomain is an anti-EPCAM antibody.

6. The method of claim 1, wherein the cancer cell antigen is a HER-2 receptor, and wherein the recombinant protein ectodomain is an anti-HER-2 antibody.

7. The method of claim 1, wherein the recombinant protein transmembrane domain further comprises a transmembrane domain selected from the group consisting of glycolipid-linked domains, CD8-based transmembrane domains, CD4-based transmembrane domains, TLR-based transmembrane domains, and combinations thereof.

8. The method of claim 1, wherein the recombinant protein further comprises an antigen presenting cell recruiting domain, wherein the recruiting domain directs the antigen presenting cells towards the cancer cells, wherein the recruiting domain is positioned on the recombinant protein ectodomain, wherein the recruiting domain comprises a receptor, and wherein the receptor directs the antigen presenting cells to cancer cells that secrete a protein that binds to the receptor.

9-10. (canceled)

11. The method of claim 8, wherein the receptor is CCR6, and wherein secreted protein is CCL20.

12. The method of claim 9, wherein the receptor is an IL-10 receptor, and wherein the secreted protein is IL-10.

13. The method of claim 1, wherein the recombinant protein further comprises an antigen presenting cell activator, wherein the antigen presenting cell activator activates or licenses the antigen presenting cells, wherein the antigen presenting cell activator is positioned on a recombinant protein endodomain, and wherein the antigen presenting cell activator activates the antigen presenting cells through upregulation of the expression of proteins selected from the group consisting of major histocompatibility complex proteins, co-stimulatory proteins, pro-inflammatory cytokines, toll-like receptors, or combinations thereof.

14-15. (canceled)

16. The method of claim 13, wherein the antigen presenting cell activator is selected from the group consisting of CD40, TLR4, TLR2, TLR3, TLR7, TLR8, TLR9, TLR5, FLT3, 4-1BB, LTBR, RANK, and combinations thereof.

17. The method of claim 13, wherein the antigen presenting cell activator is at least one of CD40 and TLR4, and wherein the antigen presenting cells are activated after signal transduction by at least one of CD40 and TLR4 to upregulate expression of MHC II, CD80, co-stimulatory protein CD86, pro-inflammatory cytokines, or combinations thereof.

18-21. (canceled)

22. The method of claim 1, wherein the carrier comprises nanoparticles.

23. The method of claim 1, wherein the nucleotide sequence is selected from the group consisting of DNA, DNA on a plasmid, mRNA, and combinations thereof.

24. (canceled)

25. The method of claim 1, wherein the antigen presenting cells are selected from the group consisting of macrophages, B-cells, dendritic cells, and combinations thereof.

26. (canceled)

27. The method of claim 1,
wherein the introduction of the carrier into the antigen presenting cells occurs in vitro, and wherein the antigen presenting cells are administered to a subject after the introduction; or
wherein the introduction of the carrier into the antigen presenting cells occurs in vivo in a subject, wherein the introduction of the carrier into the antigen presenting cells occurs by administration of the carrier to the subject, and wherein the administration results in the preferential uptake of the carrier by the antigen presenting cells of the subject.

28-29. (canceled)

30. The method of claim 27, wherein the subject is suffering from cancer, wherein the method is utilized to treat the cancer, and wherein the antigen presenting cells enable the immune system to attack the cancer cells of the subject.

31. The method of claim 30, wherein the cancer is selected from the group consisting of breast cancer, gastrointestinal carcinomas, head and neck cancer, hepatocellular carcinoma, lung cancer, ovarian cancer, pancreatic cancer, leukemia, multiple myeloma, and combinations thereof.

32. (canceled)

33. The method of claim 30,
wherein the antigen presenting cells comprise dendritic cells,
wherein the antigen presenting cells enable T-cells to attack the cancer cells by binding to the cancer cells through interaction between the recombinant protein ectodomain of the antigen presenting cells and the antigen of the cancer cells,
wherein the interaction results in the presentation of the antigen of the cancer cells on a surface of the antigen presenting cells,
wherein the antigen presenting cells present the antigen of the cancer cells on the surface of the antigen presenting cells to the T-cells, and
wherein the T-cells are activated to initiate anti-cancer immune responses against the cancer cells.

34. (canceled)

35. The method of claim 33, wherein the antigen presenting cells comprise macrophages,
wherein the macrophages bind to the cancer cells through interaction between the recombinant protein ectodomain of the macrophages and the antigen of the cancer cells,
wherein the interaction results in the killing of the cancer cells by the macrophages, and
wherein the killing of the cancer cells by the macrophages occurs by phagocytosis of the cancer cells by the macrophages.

36-81. (canceled)
